(19) United States
(12) Patent Application Publication
(10) Pub. No.: US 2008/0145423 A1
(43) Pub. Date: Jun. 19, 2008

(54) Chewable tablet and method of formulating

(76) Inventors: Ajmal Ali Khan, Germantown, TN (US); Eddie Brunson, Memphis, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/639,016

(22) Filed: Dec. 14, 2006

(51) Int. Cl. A61K 9/20; B27N 3/00

(52) U.S. Cl. 424/465; 264/109

(57) ABSTRACT

Processes for preparing a chewable tablet comprising a micronized form of an active ingredient, the method comprising the steps of combining the active ingredient with tablet excipients by geometric dilution to form a final mixture and applying direct compression to at least a portion of the final mixture to form at least one tablet.

### BACKGROUND OF THE INVENTION

Chewable tablet formulations, particularly those containing pharmaceutically active agents, present issues of organoleptic characteristics of odor, taste, appearance and mouth feel. The formula ingredients and manufacturing process both play a role in obtaining the desired organoleptic properties.

Manufacturing of tablet products is generally done using either a wet granulation process or direct compression, see, e.g., Pharmaceutical Dosage Forms: Tablets Volume 1, Marcel Dekker Inc. Second Edition 1989, editors Lieberman, Lachman and Schwarz, Page 131 and 195. The wet granulation process typically involves wet massing of the formula ingredients using a liquid to form aggregates. The process requires a drying step to remove the liquid, following which the dried aggregates are reduced to an appropriate size by milling. Over-wetting of granules in the wet granulation process can produce harder granules. Tablets made from such granulations often have a gritty mouth-feel when chewed, see, e.g., Pharmaceutical Dosage Forms: Tablets Volume 1, Marcel Dekker Inc. Second Edition 1989, editors Lieberman, Lachman and Schwarz, Page 396. This grittiness can be avoided by using a direct compression manufacturing process which eliminates the wet massing and subsequent drying steps.

Increasingly, micronized and submicron forms of therapeutically and/or physiologically active substances are being incorporated into tablet formulations to take advantage of the enhanced absorption characteristics of these forms. However, when the amount of an active substance contained in the tablet is low, the use of a micronized or submicron form makes it difficult to disperse the drug uniformly in the powder blend using a conventional direct compression process.

Thus, a method of preparing a chewable tablet that comprises a low concentration of micronized or submicron active ingredient forms but having a uniform active ingredient dispersion and beneficial organoleptic qualities would be desirable.

These and other benefits are achieved by the present invention, which provides a direct compression manufacturing process that involves geometric dilution of micronized active ingredient with other excipients. In the process of the invention, the addition sequence of formula ingredients, preparation of premixes of the formula ingredients and milling or screening steps at various stages of the manufacturing process allows uniform distribution of active ingredient in the powder mix, while preserving the compressibility characteristics of the excipients.

### SUMMARY OF THE INVENTION

The present invention provides a process for preparing a chewable tablet comprising a micronized form or submicron form of an active ingredient, the method comprising the steps of combining the active ingredient with tablet excipients by geometric dilution to form a final mixture and applying direct compression to at least a portion of the final mixture to form at least one tablet. In a preferred embodiment, at least one of the active ingredients present in the composition is an antihistamine. In particularly preferred embodiments, the active ingredient is loratadine or desloratadine.

The invention also provides a process for preparing a chewable tablet from a composition comprising an amount of at least one active ingredient in micronized form or submicron form and a plurality of tableting excipients, wherein the chewable tablet exhibits acceptable content uniformity of active ingredient, the process comprising:

a) dividing a first tableting excipient into a plurality of portions;

b) combining a percentage of the amount of the active ingredient with a first portion of the first tableting excipient in the absence of additional tableting excipients to form a primary premixture;

c) combining one or more additional tableting excipients from the plurality of tableting excipients into one or more remaining portions of the first tableting ingredient not containing the active ingredient to form one or more secondary premixtures;

d) adding said one or more secondary premixtures to the primary premixture to form a main batch; and

e) applying direct compression to at least a portion of the main batch to form at least one tablet.

In the practice of the process, the percentage of the amount of the active ingredient added to said first portion of said first tableting excipient is typically greater than 50%, and in additional embodiments can be greater than 75%, greater than 80%, greater than 90%, up to 100%. In the practice of the process wherein less than the entire amount of active ingredient is added to the first portion, the remainder of the amount of active ingredient is added to one or more of the secondary premixtures prior to formation of the main batch. In the practice of the process of this aspect of the invention, the so called first tableting excipient is typically, but not always, the largest component of the composition by weight percentage of the total composition. In one embodiment of the process according to the invention, the first tableting excipient is a sweetener, such as mannitol. However, as described herein, additional tableting excipients can be used in the compositions of the invention.

In additional embodiments of the practice of the process of the invention, the secondary premixtures can be separately added to the primary premixture or two or more of said secondary premixtures can be combined prior to being added to the primary premixture.

In separately preferred embodiments of the process of the invention, one or more of the tableting excipients can be deagglomerated during the process, such as by passing through a mill or screen. As equally preferred examples, the first tableting excipient can be deagglomerated prior to step (a) or step (b). Further, by way of example, one or more of the primary premixture and the one or more secondary premixtures can be deagglomerated prior to step (d).

The process of the invention may further comprise the step of combining one or more tableting excipients with at least one portion of said main batch to form a tertiary premixture and then adding that tertiary premixture to the main batch. In one embodiment of this aspect of the invention the tertiary premixture may be deagglomerated, such as by passing through a mill or screen, prior to its being added to said main batch.

The invention further provides a process for preparing a chewable tablet comprising an antihistamine in micronized form or submicron form and tableting excipients, the method comprising the steps of combining antihistamine with tableting excipients by geometric dilution to form a final mixture and applying direct compression on the final mixture to produce tablet shapes, wherein any two of said chewable tablets exhibits bioequivalence to one non-chewable tablet comprising an equal or similar amount of antihistamine. In equally preferred embodiments, the antihistamine is loratadine or desloratadine.

The invention also provides a chewable tablet comprising an antihistamine in micronized form or submicron form and tableting excipients, wherein the chewable tablet exhibits acceptable organoleptic qualities, antihistamine content uniformity, rapid dissolution, a substantial absence of binding in the die cavities and a substantial absence of sticking to punch faces under compression. In a preferred embodiment, two of said chewable tablets exhibit bioequivalence to one nonchewable tablet comprising an equal or similar amount of antihistamine. In a preferred embodiment of the invention, the antihistamine is present in an amount up to about 2.0 weight % per tablet. In equally preferred embodiments, the antihistamine is loratadine or desloratadine.

### BRIEF DESCRIPTION OF THE DRAWINGS

FIG. 1 provides an example of an embodiment of a manufacturing process according to the invention.

### DETAILED DESCRIPTION

The term active ingredient, as used herein, refers any substance having a measurable activity of therapeutic, cosmetic or nutraceutical nature, towards a human or animal to which the active ingredient is administered. One or more of numerous such active ingredients can be utilized in forming the products of this invention, including, for example, pharmaceuticals, dietary supplements, animal feeds, or biocidal agents. The processes according to the present invention are particularly useful for preparation of tablets comprising low amounts of micronized or submicron active ingredients, such as formulations containing up to about 5% by weight of the tablet, alternatively containing up to about 4%, about 3%, about 2%, or about 1% by weight of the tablet.

Where the active ingredient is a pharmaceutical agent, representative general classifications of such agents include, for example, adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormonal sterol derivatives; plasminogen activators; platelet activating factor antagonists; platelet aggregation inhibitors; psychotropics; radioactive agents; scabicides; sclerosing agents; sedatives; sedative-hypnotics; selective adenosine AI antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; xanthine oxidase inhibitor; and anti-cancer agents such as taxol and paclitaxel.

Examples of analgesics include codeine, diamorphine, dihydromorphine, ergotamine, fentanyl and morphine; examples of antiallergics include cromoglycic acid and nedocromil; examples of antibiotics include cephalosporins, fusafungin, neomycin, penicillins, pentamidine, streptomycin, sulfonamides and tetracyclines; examples of anticholinergics include atropine, atropine methonitrate, ipratropium bromide, oxitropium bromide and trospium chloride; examples of antihistamines include H₁ or H₂ antagonists or other types of histamine release inhibitors, the H₁ antagonists can be sedating or non-sedating, such as diphenhydramine, chlorpheniramine, tripelennamine, promethazine, clemastine, doxylamine, astemizole, terfenadine, loratadine and desloratadine, among others, the H₂ antagonists include, but are not limited to, cimetadine, famotidine, nizatidine, and ranitidine; examples of histamine-release-inhibitors include, but are not limited to, cromolyn; examples of antiinflammatory substances include beclomethasone, budesonide, dexamethasone, flunisolide, fluticasone, tipredane and triamcinolone; examples of antitussives include narcotine and noscapine; examples of bronchodilators include bambuterol, bitolterol, carbuterol, clenbuterol, ephedrine, epinephrine formoterol, fenoterol, hexoprenaline, ibuterol, isoprenaline, isoproterenol, metaproterenol, orciprenaline, phenylephrine, pseudoephedrine, phenylpropanolamine, pirbuterol, procaterol, reproterol, rimiterol, salbutamol, salmeterol, sulfonterol, terbutalin and tolobuterol; examples of diuretics include amiloride and furosemide; examples of enzymes include amylase, lipase, protease and trypsin; examples of cardiovascular substances include diltiazem and nitroglycerine; examples of hormones include cortisone, hydrocortisone, prednisolone cyproterone acetate, norethisterone acetate, progesterone, 3-keto-desogestrel, norgestimate, laevonorgestrel, desogestrel, gestodene, estrogen, δ-4-androstenedione, testosterone, dihydrotestosterone, or androstanolone, examples of proteins and peptides include cyclosporins, cetrorelix, glucagon and insulin.

The active ingredients mentioned by way of example can be employed as free bases or acids or as pharmaceutically acceptable salts. Counterions which can be employed are, for example, physiologically tolerable alkaline earth metals or alkali metals or amines, as well as, for example, acetate, benzenesulfonate, benzoate, hydrogen carbonate, hydrogen tartrate, bromide, chloride, iodide, carbonate, citrate, fumarate, malate, maleate, gluconate, lactate, pamoate and sulfate. Esters can also be employed, for example including but not limited to acetate, acetonide, propionate, dipropionate, valerate.

The term “micronized” as used herein has its customary meaning, and generally refers to materials having a particle size ranging from about 1 μm up to about 30 μm, more preferably up to about 20 μm and more preferably up to about 5 μm. The term “submicron” as used herein has its customary meaning, and generally refers to materials having a particle size up to about 1 μm. Methods of making micronized and submicron forms of active ingredients are well known in the art and include, for example, attrition mills, fluid energy mills and micronizers. Methods are discussed, for example, in Advanced Pharmaceutical Solids, Jens T. Cartensen. p 330 (Marcel Dekker Inc. 2001) and Rasenack, M. and Muller, B. W., “Micron-Size Drug particles: Common and Novel Micronization Techniques”, Pharmaceutical Development and Technology, Vol 9, No. 1, pp. 1-13 (2004).

As used herein, the term “bioequivalence” has its customary meaning referring to the ratio of the least square mean of Cₘₐₓ and AUC of a test compound to the least square mean of Cₘₐₓ and AUC of the reference compound falling within 0.8 to 1.25 at the 90% confidence intervals.

In the blending and compaction of such active ingredients, additional components, generally referred to herein as tableting excipients, can be included which have desirable functions and characteristics that enable the formation of chewable tablets comprising the micronized active ingredient. For example, tableting excipients include, but are not limited to, agents that impart desired attributes of chewablity and mouth feel, flow aids, disintegrants, lubricants, mold release agents, sweeteners and flavorants, colorants, stabilizers, adjuvants, corrosion inhibitors, dyes, surfactants, synergists, effervescents, diluents, builders, chelating agents, buffers, and the like. Those of ordinary skill in the art recognize that choice of any one tableting excipient will depend on its inherent function as well as its compatibility with the active ingredient such that it does not interfere in any material way with its performance characteristics.

As used herein, binders are those substances known to one of ordinary skill in the art as natural or synthetic binders, e.g., hydroxyalkyl cellulose, such as hydroxy methyl cellulose and hydroxy ethyl cellulose; methyl cellulose; plant gums including traganth gum, gum arabicum, carayagum, guar gum, xanthan gum, and irish moss; polyvinyl pyrrolidone, polynicyl alcohol, polyvinyl acetate, gelatin, starch, carboxy methyl starch; polyurethanes, synthetic polyelectrolytes, polyalkylene glycols, inorganic thickening agents, including various forms of silicon oxide and silicon dioxide, such as amorphous, hydrogels, pyrogenic, sublimated or suspended particles, and silicates.

Conventional adjuvants include, for example, carrier substances, such as the sugars lactose, saccharose, mannitol, or sorbitol, cellulose preparations and/or calcium phosphate, such as tricalcium phosphate or calcium hydrogen phosphate, the starches of corn, wheat, rice, or potato, methyl cellulose, hydroxy methyl cellulose, sodium carboxy-methyl cellulose and/or polyvinyl pyrrolidone. Additional adjuvants are in particular flow aids and lubricants, include such substances as silica, talcum, stearic acid or salts thereof such as the magnesium, calcium, sodium, and potassium stearates, polyethylene glycol and its derivatives, hard paraffin and combinations thereof.

Wetting agents are materials that accelerate the solubilization and/or the dissolution of the active substance(s) and the other excipients contained in the micronized powder.

Diluents are materials added to the powder containing the active substance(s) until a predetermined total volume containing a selected amount of the active substance(s) is obtained.

Sweeteners may be those typically used in consumable products and include, but are not limited to, such substances as aspartame, dextrates, dextrose, fructose, mannitol, sodium or calcium saccharinate, sorbitol, sucralose, sucrose, and mixtures thereof. The flavor additive may be any flavors of synthetic, semi-synthetic or natural origin that are typically used in consumable products and are well known to those of ordinary skill in the art. Representative examples include, but are not limited to, mint, peppermint, lemon, grape, banana, strawberry, raspberry, orange, vanilla, caramel, and mixtures thereof. Also, colorants can be added to the mixtures, including all FD&C and D&C colorants approved for use in foods, drugs, cosmetics and/or medical devices. For example, a list of colorants approved for use in foods, drugs, cosmetics and/or medical devices in the United States is provided at 21 C.F.R. Parts 73 and 74, which list is periodically updated. It is understood that similar lists in various jurisdictions, and updated versions thereof, will guide those of ordinary skilled in the art towards appropriate colorants for use in preparing compositions and tablets according to the invention. In particular embodiments of the invention, the colorant may be FD&C blue #2 dye aluminum lake, D&C red #27 dye aluminum lake, FD&C blue #1 dye aluminum lake, FD&C green #3 dye aluminum lake, D&C green #5 dye aluminum lake, FD&C red #3 dye aluminum lake, D&C red #6 dye aluminum lake, D&C red #7 dye aluminum lake, D&C red #21 dye aluminum lake, D&C red #22 dye aluminum lake, D&C red #30 dye aluminum lake, D&C red #33 dye aluminum lake, D&C red #36 dye aluminum lake, D&C red #40 dye aluminum lake, FD&C red #40 dye aluminum lake, FD&C yellow #5 aluminum lake, FD&C yellow #6 dye aluminum lake, D&C yellow #10 dye aluminum lake, synthetic iron oxide pigments and combinations thereof.

The invention will be further described by means of the following examples, which are not intended, and should not be interpreted, to limit the invention which is more fully defined by the claims which follow thereafter.

### Experimental

Chewable tablets containing loratadine were manufactured using a direct compression process according to the subject invention to produce tablets with sufficient flow and compressibility suitable for high speed manufacturing while assuring uniform loratadine content uniformity, rapid dissolution, absence of binding in the die cavities and absence of sticking to punch faces under compression. The loratadine used was micronized material added at a relatively low concentration resulting in a final content of 5 mg loratadine per tablet.

### Manufacturing Process

Chewable tablets were prepared according to the process described herein containing the following materials per tablet. The batch size was 2.1 million tablets.

TABLE 1

| Ingredient | Weight Per Tablet |
|---|---|
| Loratadine (micronized) | 5 mg |
| Aspartame | 2.50 mg |
| FD &C Blue #2 dye aluminum lake | 1.25 mg |
| 12–14% dye | |
| D&C red #2 dye aluminum lake 16–21% | 1.25 mg |
| dye | |
| Microcrystalline cellulose | 20 mg |
| Citric acid | 1.88 mg |
| Grape flavor | 1.25 mg |
| Magnesium stearate | 3.50 mg |
| Mannitol | 201.12 mg |
| Sodium starch glycolate | 7.50 mg |
| Colloidal silicon dioxide | 1.25 mg |
| Stearic acid | 3.5 mg |

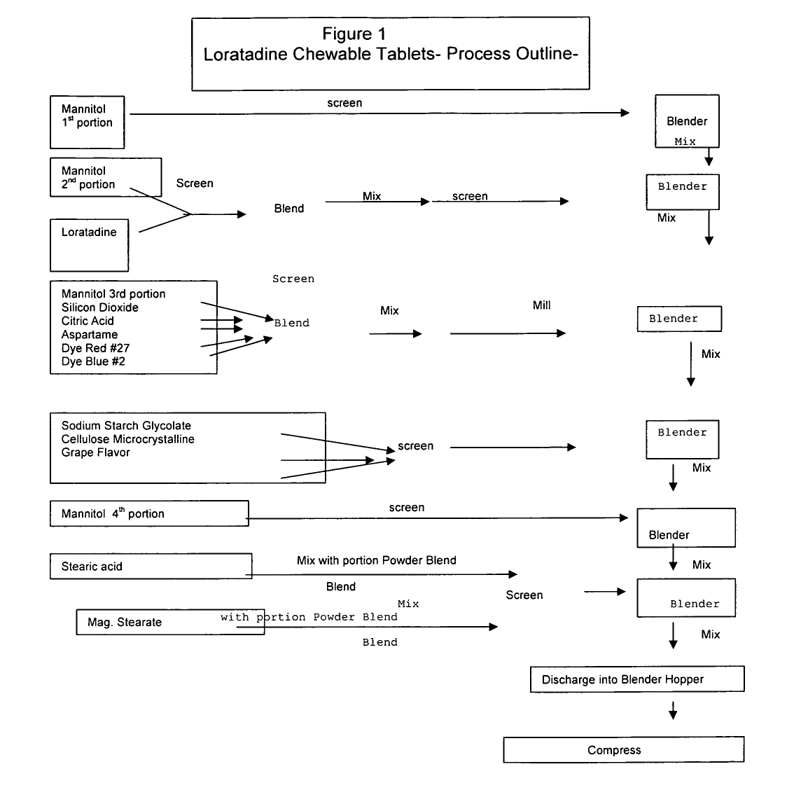

FIG. 1 provides the flow diagram of the manufacturing process used. Spray dried mannitol was de-agglomerated before use by milling and then added in four separate portions to the batch. Loratadine was dispersed in a pre-mixture with a portion of the mannitol and de-agglomerated by passing through a screen. A second pre-mixture was prepared consisting of the FD&C blue #2 aluminum lake, D&C red #27 aluminum lake, citric acid, aspartame and colloidal silicon dioxide. The pre-mixture was then de-agglomerated by passing through a mill. Sodium starch glycolate, microcrystalline cellulose and grape flavor were then passed through a screen individually before addition to the batch. The lubricants magnesium stearate and stearic acid were then mixed with portions of the material from the main batch and passed through a screen before adding to the batch. After addition of the lubricants, the powder was mixed and then compressed into tablets. The process steps for preparation of the powder mixture are done sequentially as a batch over a period of about 8 hours. The currently verified hold time for prepared powder mixture prior to compression into tablets is 14 days.

### Content Uniformity Tests

Content uniformity testing on the compressed tablets provides an assessment of how uniformly the micronized or submicron active ingredient is dispersed in the powder mixture. Content uniformity of loratadine in tablets prepared according to the invention was determined according to the following procedure: the tablet was accurately weighed and placed into a 50 mL volumetric flask and 20 mL of a diluent was then added. The diluent was prepared by adding 400 mL of 0.05N hydrochloric acid and 80 mL of 0.6M dibasic potassium phosphate into a 1.0 L volumetric flask, diluted to volume with methanol:acetonitrile (1:1) and mixed. The flasks with the tablets and diluent were shaken on a wrist action shaker for 40 minutes. The flasks were diluted to volume with the diluent and mixed. A portion of the solution was filtered through a 0.45 micron Whatman GD/X hrdrophobic PTFE membrane filter. The first three mL were discarded and then an aliquot was collected in an HPLC vial for HPLC analysis.

The following chromatographic conditions were used for HPLC analysis. Mobile phase: Phosphate buffer, pH 7.2/Methanol/Acetonitrile (50:10:40 v/v/v); flow rate: 1.5 mL/min; detector: UV @ 254 nm; injection volume 15 μL; run time: 25 minutes; column: Waters Symmetry Sheild RP8, 4.6 mm×250 mm, 5 μm.

Acceptance criteria used for content uniformity is the current USP <905> for content uniformity of tablets which consists of two stages of testing. Stage 1 testing is conducted on 10 tablets to determine percent label strength, with the goal of determining no tablet outside 85.0% to 115.0% of label strength, with relative standard deviation (RSD) less than or equal to 6.0%. If necessary, stage 2 testing would be conducted on 30 tablets with the goal of determining percent label strength within 75.0% to 125% of label strength, RSD less than or equal to 7.8%.

All batches passed at Stage 1 testing, therefore no Stage 2 testing was completed. The average content uniformity RSD for batches was less than 2.2%. The results indicate that the manufacturing process provides a high degree of dispersion of the micronized active ingredient in the direct compression manufacturing process.

### Bioequivalence Study

A randomized, open label, single dose, two-way crossover study was performed to compare the bioequivalence of 2×5 mg loratadine chewable tablets prepared according to the invention against one loratadine 10 mg swallow tablet in healthy adult subjects. A total of 48 subjects were enrolled and all 48 subjects were analyzed. Subjects were screened for eligibility within 32 days of receiving the first dose of study medication. Subjects were randomly assigned to one of two treatment sequences during two study periods (2×5 mg loratadine chewable tablets followed by 1×10 mg loratadine swallow tablet or 1×10 mg loratadine swallow tablet followed by 2×5 mg loratadine chewable tablets). A 14 day washout period separated the two doses of study medication. Subjects were confined to the study site on the day prior to the study drug administration and for 120 hours following study drug administration for collection of pharmacokinetic blood samples and safety monitoring.

Standard pharmacokinetic parameters were determined for loratadine and desloratadine:

AUC_{T}: the area under the concentration-time curve from time zero to last measurable concentration;

AUC: the area under the concentration-time curve from time zero to infinity;

Cₘₐₓ: the maximal plasma concentration;

tₘₐₓ: the time to reach Cₘₐₓ;

λ_{Z}: terminal rate constant; and

t_{1/2}:elimination half life.

### Results of the bioequivalence study are summarized in Table 2

TABLE 2
Analysis of Loratadine and Desloratadine Bioequivalence

| | | | | | |
|---|---|---|---|---|---|
| | 2 × 5 mg | 1 × 10 mg | | | |
| Analyte | tablets | tablet | Ratio | 90% CI | 90% CI |
| Parameter | Test | Reference | Test/Reference | Lower | Upper |

Loratadine

| | | | | | |
|---|---|---|---|---|---|
| AUC | 8.1305 | 7.4828 | 1.0866 | 0.9859 | 1.1975 |
| AUC_{T} | 7.5800 | 6.9576 | 1.0895 | 0.9886 | 1.2006 |
| Cₘₐₓ | 2.5926 | 2.4255 | 1.0689 | 0.9432 | 1.2114 |

Desloratadine

| | | | | | |
|---|---|---|---|---|---|
| AUC | 38.1611 | 38.3228 | 0.9958 | 0.9489 | 1.0450 |
| AUC_{T} | 36.3022 | 36.6214 | 0.9913 | 0.9425 | 1.0425 |
| Cₘₐₓ | 2.4235 | 2.6152 | 0.9267 | 0.8695 | 0.9877 |

CI = Confidence Interval

In the analysis of bioequivalence, the 90% confidence intervals around the ratio of the least square mean for loratadine and the metabolite desloratadine Cₘₐₓ and AUC fell completely within the bioequivalence intervals of 0.8 to 1.25, showing that the 2×5 mg loratadine chewable tablets were bioequivalent to the 1×10 mg loratadine tablet for loratadine and the active metabolite desloratadine.

What is claimed is:

1. A process for preparing a chewable tablet comprising a micronized form or submicron form of an active ingredient, the method comprising the steps of combining the active ingredient with tablet excipients by geometric dilution to form a final mixture and applying direct compression to at least a portion of the final mixture to form at least one tablet.

2. The process according to claim 1, wherein the active ingredient is chosen from the group consisting of adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormonal sterol derivatives; plasminogen activators; platelet activating factor antagonists; platelet aggregation inhibitors; psychotropics; radioactive agents; scabicides; sclerosing agents; sedatives; sedative-hypnotics; selective adenosine AI antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; xanthine oxidase inhibitor; anti-cancer agents, and combinations thereof.

3. The process according to claim 2, wherein the active ingredient is an antihistamine.

4. The process according to claim 3, wherein the active ingredient is loratadine or desloratadine.

5. The process according to claim 1, wherein the active ingredient is present in an amount up to about 2.0 weight %.

6. A process for preparing a chewable tablet from a composition comprising an amount of at least one active ingredient in micronized form or submicron form and a plurality of tableting excipients, wherein the chewable tablet exhibits acceptable content uniformity of active ingredient, the process comprising:
a) dividing a first tableting excipient into a plurality of portions;
b) combining a percentage of the amount of the active ingredient with a first portion of the first tableting excipient in the absence of additional tableting excipients to form a primary premixture;
c) combining one or more additional tableting excipients from the plurality of tableting excipients into one or more remaining portions of the first tableting excipient not containing the active ingredient to form one or more secondary premixtures;
d) adding said one or more secondary premixtures to the primary premixture to form a main batch; and
e) applying direct compression to at least a portion of the main batch to form at least one tablet.

7. The process of claim 6, wherein the percentage of the amount of the active ingredient added to said first portion of said first tableting excipient is greater than 50%.

8. The process of claim 6, wherein the percentage of the amount of the active ingredient added to said first portion of said first tableting excipient is greater than 75%.

9. The process of claim 6, wherein the percentage of the amount of the active ingredient added to said first portion of said first tableting excipient is greater than 80%.

10. The process of claim 6, wherein the percentage of the amount of the active ingredient added to said first portion of said first tableting excipient is greater than 90%.

11. The process of claims 7-10, wherein the remainder of the amount of active ingredient is added to one or more of the secondary premixtures.

12. The process of claim 6, wherein the percentage of the amount of the active ingredient added to said first portion of said first tableting excipient is 100%.

13. The process of claim 6, wherein said first tableting excipient is the largest component of the composition by weight percentage of the total composition.

14. The process of claim 6, wherein said secondary premixtures are separately added to the primary premixture.

15. The process of claim 6, wherein at least two of said secondary premixtures are combined prior to being added to the primary premixture.

16. The process of claim 6, wherein the first tableting excipient is de-agglomerated by passing through a mill prior to step (a).

17. The process of claim 6, wherein the first tableting excipient is de-agglomerated by passing through a mill prior to step (b).

18. The process of claim 6, wherein one or more of said primary premixture and said one or more secondary premixtures are de-agglomerated by milling prior to step (d).

19. The process of claim 6, wherein one or more of said primary premixture and said one or more secondary premixtures are de-agglomerated by passing through a screen prior to step (d).

20. The process of claim 6, further comprising the step of combining one or more tableting excipients with at least a portion of said main batch to form a tertiary premixture and then adding said tertiary premixture to said main batch.

21. The process of claim 20, wherein said tertiary premixture is passed through a screen prior to its being added to said main batch.

22. The process of claim 6, wherein the tableting excipients are chosen from the group consisting of agents that impart desired attributes of chewablity and mouth feel, flow aids, disintegrants, lubricants, mold release agents, sweeteners and flavorants, colorants, stabilizers, adjuvants, corrosion inhibitors, dyes, surfactants, synergists, effervescents, diluents, builders, chelating agents, buffers, and mixtures thereof.

23. The process according to claim 6, wherein the first tableting excipient is a sweetener.

24. The process according to claim 23, wherein the sweetener is chosen from the group consisting of aspartame, dextrates, dextrose, fructose, mannitol, sodium saccharinate, calcium saccharinate, sorbitol, sucralose, sucrose, and mixtures thereof.

25. The process according to claim 6, wherein the first tableting excipient is mannitol.

26. The process according to claim 6, wherein the one or more active ingredients are chosen from the group consisting of adrenergics; adrenocortical steroids; adrenocortical suppressants; aldosterone antagonists; amino acids; anabolics; analeptics; analgesics; anesthetics; anorectics; antiacne agents; antiadrenergics; antiallergics; antiamebics; antianemics; antianginals; antiarthritics; antiasthmatics; antiatherosclerotics; antibacterials; anticholinergics; anticoagulants; anticonvulsants; antidepressants; antidiabetics; antidiarrheals; antidiuretics; antiemetics; antiepileptics; antifibrinolytics; antifungals; antihemorrhagics; antihistamines; antihyperlipidemics; antihypertensives; antihypotensives; antiinfectives; antiinflammatories; antimicrobials; antimigraine; antimitotics; antimycotics, antinauseants, antineoplastics, antineutropenics, antiparasitics; antiproliferatives; antipsychotics; antirheumatics; antiseborrheics; antisecretories; antispasmodics; antithrombotics; antiulceratives; antivirals; appetite suppressants; blood glucose regulators; bone resorption inhibitors; bronchodilators; cardiovascular agents; cholinergics; depressants; diagnostic aids; diuretics; dopaminergic agents; estrogen receptor agonists; fibrinolytics; fluorescent agents; free oxygen radical scavengers; gastrointestinal motility effectors; glucocorticoids; hair growth stimulants; hemostatic agents; histamine H₂ receptor antagonists; hormones; hypocholesterolemics; hypoglycemics; hypolipidemics; hypotensives; imaging agents; immunizing agents; immunomodulators; immunoregulators; immunostimulants; immunosuppressants; keratolytics; LHRH agonists; mood regulators; mucolytics; mydriatics; nasal decongestants; neuromuscular blocking agents; neuroprotective agents; NMDA antagonists; non-hormonal sterol derivatives; plasminogen activators; platelet activating factor antagonists; platelet aggregation inhibitors; psychotropics; radioactive agents; scabicides; sclerosing agents; sedatives; sedative-hypnotics; selective adenosine AI antagonists; serotonin antagonists; serotonin inhibitors; serotonin receptor antagonists; steroids; thyroid hormones; thyroid inhibitors; thyromimetics; tranquilizers; amyotrophic lateral sclerosis agent; cerebral ischemia agent; Paget's disease agent; unstable angina agent; vasoconstrictor; vasodilator; wound healing agent; xanthine oxidase inhibitor; anti-cancer agents, and combinations thereof.

27. The process according to claim 26, wherein the active ingredient is an antihistamine.

28. The process according claim 27, wherein two of said chewable tablets exhibits bioequivalence to one non-chewable tablet comprising an equal or similar amount of antihistamine.

29. The process according to claim 27, wherein the active ingredient is loratadine or desloratadine.

30. The process according to claim 6, wherein the active ingredient is present in an amount up to about 2.0 weight %.

31. The process according to claim 6, wherein the active ingredient is in micronized form.

32. The process according to claim 6, wherein the active ingredient is in submicron form.

33. A process for preparing a chewable tablet comprising an antihistamine in micronized form or submicron form and tableting excipients, the method comprising the steps of combining the loratadine with tableting excipients by geometric dilution to form a final mixture and applying direct compression on the final mixture to produce tablet shapes, wherein any two of said chewable tablets exhibits bioequivalence to one nonchewable tablet comprising an equal or similar amount of antihistamine.

34. The process according to claim 33, wherein the antihistamine is loratadine or desloratadine.

35. A chewable tablet prepared by the process according to claims 1, 6, and 33.

36. A chewable tablet comprising an antihistamine in micronized form or submicron form and tableting excipients, wherein the chewable tablet exhibits acceptable organoleptic qualities, antihistamine content uniformity, rapid dissolution, a substantial absence of binding in the die cavities and a substantial absence of sticking to punch faces under compression.

37. The chewable tablet according to claim 36, wherein the antihistamine is loratadine or desloratadine.

38. The chewable tablet according to claim 37, wherein the antihistamine is loratadine and the loratadine is present in an amount up to about 2.0 weight % per tablet.
